(19) United States
(12) Patent Application Publication
(10) Pub. No.: US 2005/0112214 A1
(43) Pub. Date: May 26, 2005

(54) Method for reducing acne for improving skin tone

(76) Inventors: Benjamin Wiegand, Newtown, PA (US); Laura McCulloch, Cedar Knolls, NJ (US); Rachel Grossman, Princeton, NJ (US); Lynn Halas, Ewing, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/980,395

(22) Filed: Nov. 3, 2004

Related U.S. Application Data
(63) Continuation of application No. 10/017,180, filed on Dec. 7, 2001.
Application No. 10/017,180 is a continuation-in-part of application No. 10/012,627, filed on Dec. 7, 2001.
(60) Provisional application No. 60/256,813, filed on Dec. 20, 2000.

(51) Int. Cl. A61N 1/30; A61K 31/7048; A61K 31/60; A61K 33/04; A61K 31/203

(52) U.S. Cl. 424/705; 514/29; 514/159; 514/557; 514/562; 514/559; 604/20

(57) ABSTRACT

The present invention relates to methods for (1) reducing the number and severity of acne lesions on the skin of a mammal; (2) ameliorating the inflammation of acne lesions on the skin of a mammal; (3) improving the skin tone of a mammal, (4) treating skin disorders; (5) treating stress related skin diseases; (6) reducing stress and improving the emotional well being of an acne sufferer; (7) reducing sebaceous gland output; and (8) increasing compliance of the administration of anti-acne products. The method comprises the administration of a sensory regimen in an amount effective to downregulate the activity of the hypothalamus-pituitary-adrenal (HPA) axis in combination with the administration of an anti-acne composition.

### CLAIM OF PRIORITY

This application is a continuation-in-part of U.S. patent application Ser. No. ______, entitled “Methods For Reducing Chronic Stress In Mammals” filed concurrently herewith (Attorney Docket No. JBP 570), and which claims priority to U.S. patent application Ser. No. 60/256,813, filed Dec. 20, 2000, the disclosures of which are hereby incorporated by reference.

### BACKGROUND OF THE INVENTION

1. Field of the Invention

The present invention relates to methods for (1) reducing the number and severity of acne lesions on the skin of a mammal; (2) ameliorating the inflammation of acne lesions on the skin of a mammal; (3) improving the skin tone of a mammal, (4) treating skin disorders; (5) treating stress related skin diseases; (6) reducing stress and improving the emotional well being of an acne sufferer; (7) reducing sebaceous gland output; and (8) increasins compliance of the administration of anti-acne products. The method comprises the administration of a sensory regimen in an amount effective to downregulate the activity of the hypothalamus-pituitary-adrenal (HPA) axis in combination with the administartion of an anti-acne composition.

2. Description of the Prior Art

Advances in technology in the last century have brought benefits to society but have resulted in greater prevalence of stress in the daily lives of people at all levels of society. Our stress response mechanisms have not adapted at the same pace as advancing technology. The effect of stress on health and well being is well documented in “Why Zebra's Don't Get Ulcers—An Updated Guide to Stress, Stress Related Diseases and Coping” by Robert M. Sapoisky, ISBN 0-7167-3210-6, Chapter 1, (5^{th} Edition 2000) and in “The Concepts of Stress and Stress System Disorders—Overview of Physical and Behavioral Homeostasis”, JAMA, Mar. 4, 1992, Vol. 267, No. 9. For example, it is known that chronic stress (an accumulation of acute stresses) can cause or aggravate many conditions including immunosuppression and vulnerability to infectious diseases, gastric conditions, sleep problems, depression, premature birth in expectant mothers, low birth weight, degeneration of brain neurons leading to memory and learning problems, elevated blood pressure, heart complications and stroke due to elevated blood lipid levels and other health complications.

While stress has a major effect on systemic health conditions, it also has been proposed to play a role in one's skin condition. Skin diseases such as psoriasis, atopic dermatitis, itch, blotchiness, poor wound repair have all been proposed to be linked to stress in some way. Some relevant references include Buske-Kirschbaum et al., Psychosomatic Medicine (1997), 59: 419-426; Denda et al., Am. J. Physiol. Regulatory Integrative Comp. Physiol. 278: R367-R372, (2000); Dhabhar et al., Proc. Natl. Acad. Sci., 1999, 96:1059-1064; Kabat-Zinn et al., Psychosomatic Medicine (1998) 60: 625-632; Tausk et al., Arch. Dermatol., 2001 137: 78-82; Kiecolt-Glaser et al., Journal Behavioral Medicine 1986 9:5-21, Garg et al., Arch. Dermatol. 2001 137: 53-59; Slominski et al., Physiological Reviews, 2000 80: 979-1020; Panconesiet et al., Psychodermatology, 1996: 399-421; Scholzen, et al., Exp. Dermatol. 1998 81-96, the disclosures of which are hereby incorporated by reference.

Another skin condition potentially linked to stress is acne vulgaris. Acne vulgaris is a skin condition that affects over 85% of all people. The following are four primary factors that lead to the formation of acne vulgaris; (1) increased sebum output resulting in oily, greasy skin; (2) increased bacterial activity normally due to an overabundance of Propionibacterium acnes; (3) plugging (hypercornification) of the follicle or pilosebaceous duct; and (4) production of inflammation by substances leaking into the dermis which cause inflammatory reactions. A more detailed description can be found in Cunliffe, William J., Acne, London: Martin Duitz Ltd., 1989, Chapter 1, which is incorporated by reference.

These primary factors can be affected by some degree by varying hormone levels in people. One of the major reasons why acne is prevalent among the teen-age populations is the dramatic increase in androgen output amongst that population which leads to increase sebum production. In addition, as hormone levels change in females during different times of their menstrual cycle, there is also a potential correlation with onset of acne lesions tied into their menstrual cycle.

Stress, which is present among both genders and all age groups, but clearly present among the teen-age and 20 -30 year old populations, also can exacerbate and lead to acne problems via a number of different pathways. For example, chronic stress can lead to a reduction in the overall immune system, which would affect the skin's ability to fight off the Propionibacterium acnes bacteria.

It is known that a functioning immune system contributes to overall good health, including healthy skin. Researchers at Shiseido and Harvard Medical School and Massachusetts General Hospital, see, for example, www.shiseido.co.jp have hypothesized that use of make-up promotes a positive state of mind which in turn promotes beautiful skin through stimulation of the nervous system and internal secretions. Conversely they hypothesize that skin condition can deteriorate because of stress.

While a recognized benefit of the use of cosmetic and beauty products is that they can contribute to a more positive mental outlook, in general, use of cosmetic and beauty products has not been demonstrated to have a significant effect on the activity of the HPA axis to a degree wherein the quality of life an individual is improved. Indeed products that are intended to resolve a skin condition typically contain benefit agents that improve the skin condition by treating the outer layers of the skin. In addition, most products are either fragrance-free or lightly fragranced, which again would limit the product's ability to deliver any type of emotional benefit through sensory stimuli.

It is a common perception that emotional stress negatively affects skin condition. See for example, Journal of Investigative Dermatology Vol. 114, No. 4, April 2000 Abstract No. 415, p. 820 which is hereby incorporated by reference. In this abstract the researchers investigated the exacerbation of acne by emotional stress through looking at the effect of secretion of a neuropeptide (“substance P”) secreted from nerve ending in response to emotional stress. In immunohistochemical studies in which they incubated human facial skin with substance P, they found expression of leukocytes (ELAM-1) and expression of a neuropeptide-degrading enzyme (NEP) in sebaceous glands. These findings imply that substance P could result in neurogenic inflammation and an effect on the sebaceous glands, such that acne may be exacerbated.

Other hormones are secreted in response to stressors which can be used as an indicator of stress. For example, the region in the brain known as the hypothalamus drives the activity of the mammalian stress response. Specifically, the hypothalamus drives the production of “stress hormones” including catecholamines and glucocorticoids. The hypothalamus responds to a stressor by activating the sympathetic nerve endings in the adrenal medulla to produce adrenaline. The hypothalamus produces corticotrophin-releasing hormone (“CRH”) which acts upon the pituitary to release adrenocorticotrophic hormone (“ACTH”) which in turn acts upon the adrenal cortex to promote the production of cortisol. The CRH and sympathetic systems participate in a positive feedback loop so that activation of one system activates the other. Since increased cortisol secretion is an indication that the HPA axis has been activated, conversely, a decrease in cortisol secretion would indicate a downregulation of HPA axis activity.

As discussed above, it is desirable to have a sustained reduction of stress related hormones in order to reduce acne, improve skin tone, and have a better quality of life. Although there are over the counter commercially available anti-acne agents for topical use, such as salicylic acid, sulfur, lactic acid, glycolic acid, pyruvic acid, urea, resorcinol, N-acetylcysteine, retinoic acid, benzoyl peroxide, octopirox, triclosan, azelaic acid, phenoxyethanol, phenoxypropanol, flavinoids, derivatives thereof, and combinations thereof, they tend to be slow acting and may have negative side effects. In general, current products are effective in reducing the clinical observation of acne but since it does not completely eliminate it the consumer is not completely satisfied with the treatment of their condition. Topical medication may also result in other undesirable effects such as irritation and dryness. Accordingly, there remains a need to improve the effectiveness of currently available commercial acne products.

In addition to the products that are available over the counter, there are also a number of different pharmaceutical treatments that have been developed for the treatment of acne. These include, but are not limited to both topical and oral agents, for example isotretinoin and tretinoin, adapalene, tazarotene, azelaic acid, minocycline, doxycycline, antibacterials such as erythromyacin and clindamycin, vitamins such as zinc, folic acid and nicotinamide, as well as combinations of these antibacterials with over the counter anti-acne agents. However, there is a continuing desire to increase the effectiveness of these pharmaceutical compounds and to improve the user's quality of life.

This invention will solve the problem of improving acne and acne related conditions through use of sensory stimuli in combination with the use of an anti-acne agent. The methods according to the present invention reduce emotional stress, downregulate the HPA axis and improve the quality of life of the acne patient.

### SUMMARY OF THE INVENTION

The present invention relates to a method for reducing the number and severity of acne lesions on the skin of a mammal. The method comprises the step of administering a sensory regimen in an amount effective to downregulate the activity of the HPA axis of said mammal in combination with the administration of an anti-acne composition comprising an effective amount of an anti-acne agent.

In other embodiments, the invention relates to methods for ameliorating the inflammation of acne lesions on the skin of a mammal and methods for improving the skin tone of a mammal using the method described above.

In yet another embodiment, the invention relates to a method for the treatment of skin disorders comprising the step of administering a sensory regimen in an amount effective to downregulate the activity of the HPA axis of said mammal in combination with the administration of an anti-acne composition. Preferably, the method can provide at least at least two of the following treatments: (a) reducing the number and severity of acne lesions on the skin of a mammal; (b) ameliorating the inflammation of acne lesions on the skin of a mammal; and (c) improving the skin tone of a mammal.

The invention also relates to a method for the treatment of stress-related skin diseases such as atopic dermatitis, sebhorreic dermatitis, psoriasis, itch, wound healing, fine lines, wrinkles, pigmentation, and combinations thereof. The method comprises the step of administering a sensory regimen in an amount effective to downregulate the activity of the HPA axis of said mammal in combination with the administration of an active agent. As used herein, the term “active agent” means therapeutic agents known in the art to be useful in the treatment and or amelioration of the symptoms of these skin conditions.

In yet another embodiment, the invention also relates to a method for reducing stress and improving the emotional well being of an acne sufferer. The method comprising the step of administering a sensory regimen in an amount effective to downregulate the activity of the HPA axis of said mammal in combination with the administration of an anti-acne composition comprising an effective amount of an anti-acne agent.

The invention also relates to a method for reducing the visual appearance of shine. The method comprises the step of administering a sensory regimen in an amount effective to downregulate the activity of the HPA axis of said mammal.

In yet another embodiment, the invention relates to a method for reducing sebaceous gland output comprising the step of administering a sensory regimen in an amount effective to downregulate the activity of the HPA axis of said mammal in combination with the administration of an anti-acne composition comprising an effective amount of an anti-acne agent.

In another embodiment, the invention relates to a method for increasing compliance of anti-acne products comprising the step of administering a sensory regimen in combination with the administration of an anti-acne composition.

### DETAILED DESCRIPTION OF PREFERRED EMBODIMENTS

As discussed above, the methods according to the present invention can be used to (1) reduce the number and severity of acne lesions on the skin of a mammal; (2) ameliorate the inflammation of acne lesions on the skin of a mammal; (3) improve the skin tone of a mammal, (4) treat skin disorders; (5) treat stress related skin diseases; (6) reduce stress and improve the emotional well being of an acne sufferer; (7) reduce sebaceous gland output; and (8) increase compliance of the administration of anti-acne products. The method comprises the step of administering a sensory regimen in an amount effective to downregulate the activity of the HPA axis of said mammal in combination with the administration of an anti-acne composition comprising an effective amount of an anti-acne agent.

As used herein, “mammals” include any of a class of warm-blooded higher vertebrates that nourish their young with milk secreted by mammary glands and have skin usually more or less covered with hair, and non-exclusively includes humans, dogs and cats.

The term “effective amount” refers to the duration of the sensory regimen sufficient to create the desired response, i.e., reduction or down-regulation of the activity of the HPA axis and reduction of total daily adrenocortical hormone. The effective amount will vary with the age, physical, and emotional condition of the mammal being treated, the nature of concurrent therapy, the specific regimen employed, and like factors.

The sensory regimen can be any regimen that is relaxing to the user. Generally, the sensory regimen is selected from the group consisting of auditory stimuli, visual stimuli, tactile stimuli, gustatory stimuli and olfactory stimuli, and combinations thereof.

Suitable auditory stimuli include, but are not limited to, music and sounds of nature that are soothing or relaxing to the consumer. The term music is used herein to include instrumental and lyrical compositions; tunes; melodies; harmonies; songs; beats and frequencies such as those from metronomes, tuning forks, bells, beat machines, chimes; poetry and rhymes. The music may be of any genre, including, but not limited to, classical, soft rock, easy listening, progressive, country, and show tunes. The sounds of nature include, but are not limited to, animal sounds, such as whales singing or birds chirping; insect sounds, such as crickets; and sounds of the environment, such as a running stream or a waterfall. Sounds that have consistently soft dynamics with minimal melodic and harmonic variability, having little or no conventional beat pitch, little or no vocal, slow tempo, little or no percussion or strong rhythym are particularly effective in relaxing or soothing the user. Sounds that use a binaural beat created by using two pure frequencies, usually one in each ear, are useful in improving the mood of the user. Binaural beats in the frequency range of delta, theta and alpha brain wave frequencies are useful for relaxing the user and beats in the frequency range of beta wave activity are useful for promoting mental alertness in the user. The auditory stimuli may include, but are not limited to, a cassette tape, video tape, compact disc, interactive toys and games, websites, and a computer audio file.

The visual stimuli may include, but are not limited to, soft lights, candles, videos, movies, paintings, murals, books, landscapes, interactive toys and games, websites, and computer image files that are soothing or relaxing to the consumer. The soft lights may be of any color, such as blue, green, pink, purple, and the like. Cool colors, such as blue and green hues, are preferred to soothe the user and aid relaxation; and warmer colors, such as oranges and reds are preferred to uplift the user. Pastel shades, which are low saturation hues, are useful in soothing the user. The light may be provided in the kit as a bulb, which can be inserted into a lamp at home, or may be provided in the kit as a lamp. Lights that utilize fiber optics may also be useful in the kits of this invention. The fiber optic lights may, as is known in the art, change colors intermittently. Soft lighting of approximately 500 lux is useful in relaxing the user, particularly in the evening hours prior to bedtime. Bright light of around 2000 lux or greater is useful in improving the mood of the user when used in the wakeful period of the day such as at awakening or any other time during the day prior to the few hours preceding bedtime.

Combinations of light and sound that have frequency patterns in the range of deita, theta and alpha brain wave frequencies are useful for relaxing the user and those that have patterns in the frequency range of beta wave activity are useful for promoting mental alertness in the user.

The tactile stimuli useful in the present invention includes, but is not limited to, computer software, interactive toys and games, bubble baths, lotions, and personal care compositions. “Personal care compositions” refers to personal cosmetic, toiletry, and healthcare products such as wipes, washes, baths, shampoos, gels, soaps, sticks, balms, sachets, pillows, mousses, sprays, lotions, creams, cleansing compositions, powders, oils, bath oils and other bath compositions which may be added to a bath. Personal care compositions may also include, but are not limited to, aerosols, candles, and substances that may be used with vaporizers. The aforementioned wipes, washes, baths, shampoos, gels, soaps, sticks, balms, sachets, pillows, mousses, sprays, lotions, creams, cleansing compositions, oils, bath oils, aerosols, candles and substances which may be used with vaporizers are commercially known to those who have a knowledge of preparing personal care compositions. One example of a suitable personal care composition is Johnson's Bedtime Bath®.

The computer software may be of an interactive nature, such that the consumer relaxes while utilizing the software. Such software includes video games, crossword puzzles and the like.

Gustatory experiences also help reduce stress. Therefore, the method of the invention may include food and beverages, such as, but not limited to, fruits, candies, crackers, cheese, teas, and the like.

The method of the invention may also include olfactory sensory experiences, such as fragrances. Fragrances that the user finds pleasant and to have a calming effect on their mood are useful in the practice of this invention. Suitable fragrances include relaxing fragrances, but are not limited to those perfume compositions described in UK application 0031047.4 the disclosure of which is hereby incorporated by reference. Also suitable are the fragrances described in co-pending U.S. patent application Ser. No. 09/676,876, filed Sep. 29, 2000 entitled “Method For Calming Human Beings Using Personal Care Compositions”, the disclosure of which is hereby incorporated by reference. Generally, the fragrance can be any fragrance that is perceivable and relaxing to the user and will downregulate the activity of the HPA axis. When the fragrance is present in the anti-acne composition, the stability of the fragrance in the presence of the anti-acne agent should be such that it remains perceivable and relaxing to the user.

It is desirable to combine multiple sensory experiences useful for downregulating HPA activity and consequently reduce adrenocortical hormone below a baseline level. For example, a daily regime may include a fragrance; soft light; bubble bath containing fragrance; and relaxing music. The fragrance may be sniffed intermittently during the day while sitting in a softly lit room and listening to the relaxing music. The bubble bath containing fragrance may be used in the morning or at night when bathing or showering while listening to the relaxing music.

In a particularly prefered embodiment, the sensory regimen is administered daily for at least one week and comprises smelling a relaxing fragrance while soaking in a bath and listening to relaxing music. Further benefits are noticed when the sensory regimen includes soft lighting as described above.

The anti-acne compositions useful in the methods according to the invention include compositions containing anti-acne agents known in the art. Suitable anti-acne agents include, but are not limited to, salicylic acid, sulfur, lactic acid, glycolic acid, pyruvic acid, urea, resorcinol, N-acetylcysteine, retinoic acid, benzoyl peroxide, octopirox, triclosan, azelaic acid, phenoxyethanol, phenoxypropanol, flavinoids, derivatives thereof, and combinations thereof. Benzoyl peroxide and salicylic acid are preferred anti-acne agents. These anti-acne agents are used in amounts well known in the art for treatment of acne. For example, salicylic acid is generally used in amounts ranging from about 0.5 to about 2% by weight of the composition. When benzoyl peroxide is used it is generally present at from about 2.5% to about 10% by weight of the composition.

In addition to the products that are available over the counter, there are also a number of different pharmaceutical treatments that have been developed for the treatment of acne which are useful in the methods according to the invention. These include, but are not limited to both topical and oral agentsisotretinoin, adapalene tretinoin, tazarotene and azelaic acid. The pharmaceutical anti-acne compounds are used in a dosing amount that is in accordance with the prescribed directions of the physician. While oral isotretinoin is effective for treating acne conditions, it is also know to have negative effects, such as depression, accordingly administration of isotretinoin in combination with the sensory regimen according to invention would be particularly beneficial.

In another embodiment of the invention, antibacterial agents known to be useful for treating acne and other skin disorders can be incorporated into the anti-acne compositions in combination with the anti-acne agents described above. Examples of suitable antibacterial agents include, but are not limited to, erythromycin and clindamycin. Generally, the antibacterial agent is used in amount that is in accordance with the prescribed directions of the dermatologist.

In the method of the invention, the anti-acne compositions are utilized as directed on the products, usually by topical application to the affected area several times daily. The effectiveness of the anti-acne composition is significantly improved through combining the use of sensory regimen with the topical application of the anti-acne agents.

As discussed above, it has been discovered that the administration of the above described sensory regimen in combination with the administration of the above described anti-acne composition can be used can be used to (1) reduce the number and severity of acne lesions on the skin of a mammal; (2) ameliorate the inflammation of acne lesions on the skin of a mammal; and (3) improve the skin tone of a mammal. Further, the reduction of acne, the amelioration of inflmmation of acne lesions and/or improved skin tone resulting from the inventive methods leads to an improved quality of life. Accordingly, in another embodiment the methods of the invention can be used for reducing stress and improving the emotional well being of an acne sufferer, the method comprising the step of administering a sensory regimen in an amount effective to downregulate the activity of the HPA axis of said mammal in combination with the administration of an anti-acne composition. In one embodiment, the stress is chronic stress.

In another embodiment, the methods according to the invention can be used for the treatment of skin disorders. As described above, the method comprises the step of administering a sensory regimen in an amount effective to downregulate the activity of the HPA axis of said mammal in combination with the administration of an anti-acne composition, wherein said treatment comprises at least two of the following: (a) reducing the number and severity of acne lesions on the skin of a mammal; (b) ameliorating the inflammation of acne lesions on the skin of a mammal; and (c) improving the skin tone of a mammal.

The methods according to the invention can also be used for the treatment of stress-related skin diseases selected from the group consisting of atopic dermatitis, sebhorreic dermatitis, psoriasis, itch, wound healing, fine lines, wrinkles, pigmentation, and combinations thereof, comprising the step of administering a sensory regimen in an amount effective to downregulate the activity of the HPA axis of said mammal in combination with the administration of an active agent. In one embodiment, the stress-related skin disease is caused by chronic stress.

In another embodiment, the invention relates to a method for reducing the visual appearance of shine on human skin comprising the step of administering a sensory regimen in an amount effective to downregulate the activity of the HPA axis of the human. Such a method may further comprise the administration of an anti-acne composition.

It has also been discovered that the administration of the above described sensory regimen in combination with the administration of the above described anti-acne composition can be used can be used to reduce sebaceous gland output. Finally, because the administration of the sensory regimen is asethically pleasing to the user, the methods according to the invention can be used to increase compliance of the administration of anti-acne products.

Examples are set forth below to further illustrate the nature of the invention and the manner of carrying it out. However, the invention should not be considered as being limited to the details thereof.

### EXAMPLES

### Example 1

### Effect of Relaxing Fragrance on Skin Condition

Two groups of panelists (11 people per group) having mild to moderate acne (≧7 inflamed lesions, ≧5 non-inflamed lesions, and ≦2 nodules on the face) participated in a study to determine the effect of the addition of a relaxing fragrance in a benzoyl peroxide skin cream which they applied to their face daily. The basic formulation for the benzoyl peroxide skin cream used by Group I is listed below:

Benzoyl Peroxide Skin Cream #1

| Ingredient | Percentage |
|---|---|
| Laureth - 4 | 0.4% |
| Hydroxypropyl Methylcellulose | 0.2% |
| Carbomer 934P | 1.75% |
| Disodium EDTA | 0.2% |
| Sodium Hydroxide | 0.29% |
| Benzoyl peroxide (“BPO”) (75%) | 6.67% |
| Water | q.s. |

The formulation for Benzoyl Peroxide Skin Cream #2 used by Group II was made by subtracting 0.2% water from the benzoyl peroxide skin cream #1 and replacing it with 0.2% fragrance (Quest PD-1861).

On day one, the participants' faces were evaluated for severity of acne in terms of the number of lesions and the type of lesions present.

Over the next two weeks, once a day, after washing with PURPOSE™ GENTLE CLEANSING WASH, commercially available from Jobnson & Johnson Consumer Companies, Inc., the panelists in each group squeezed approximately a pea sized amount of the benzoyl peroxide skin cream into the palm of the hand and applied it to the entire facial area except eye, lip, and mouth areas. The panelists were not permitted to wash their faces for at least three hours after applying the cream.

At the end of the 2 week study, the panelists were asked what their overall preference of the benzoyl peroxide skin cream they were using was and whether they agreed to the following statements:

What was your overall preference?

1. The product treated whiteheads.
2. The product treated blackheads.
3. The product reduced the size of pimples.
4. The product made my skin feel smooth.
5. The product made my skin feel soft.
6. The product made my skin have even color.
7. The product made my skin have even texture.
8. The product was gentle to my skin.
9. The product was non-irritating to my skin.
10. The product was quickly absorbed by my skin.
11. The product did not make my skin flaky or have an ashy appearance.
12. The product reduced my skin pore size.
13. The product reduced my skin redness.
14. The product was good for dry skin.
   The scale for overall preference was 0 (poor) to 10 (like extremely).
   The scale for the statements was 1 (disagree) to 5 (strongly agree).

The results are shown in Table 1.

TABLE 1

Average Score

| Question | Group 2 | Group 1 |
|---|---|---|
| Overall Preference** | 7.3 | 5.9 |
| Treat Whiteheads*** | 3.9 | 3.4 |
| Treat Blackheads*** | 3.7 | 3.3 |
| Reduced Size*** | 4.1 | 3.5 |
| Smooth Skin* | 4.5 | 3.5 |
| Soft Skin* | 4.1 | 3.2 |
| Even Skin Color* | 3.5 | 2.7 |
| Even Skin Texture* | 3.8 | 3.1 |
| Gentle* | 4.5 | 3.5 |
| Non-irritating* | 4.3 | 3.4 |
| Quick Absorption* | 4.5 | 3.5 |
| No Flaky, Ashy Appearance* | 4.1 | 2.8 |
| Reduced Pore Size* | 3.7 | 2.9 |
| Reduced Redness*** | 3.8 | 3.3 |
| Good For Dry Skin*** | 3.5 | 3.0 |

*= 95% significance
**= 85% significance
***= 80% significance

Based on the data above, it is clear that the addition of fragrance to the benzoyl peroxide skin cream composition was perceived by the participants to significantly improve the performance of the product. Improvements were seen for every symptom that was inquired about.

### Example 2

### Effect of Various Sensory Regimens on Skin Condition

Since there was a benefit observed with the addition of a relaxing fragrance in the benzoyl peroxide skin cream containing an anti-acne agent, additional work was done to determine if additional relaxation treatments would further improve the skin condition and quality of life of the panelists. Groups 1 and 2 were identical to those in Example 1 and a third group with 12 panelists was studied where there were multiple additional relaxation elements to the regime.

The following protocol was used for Group 3: Group 3 applied the topical Benzoyl Peroxide Skin Cream #1 once per day, smelled a fragrance PD1861 supplied by Quest International while sitting and listening to relaxing music from the music CD entitled “Relax with Ocean Relaxing Surf” by Eclipse Music Group for 10 minutes three times a day, and at night took a 35° C. 15 minute bubble bath containing fragrance PD1861 supplied by Quest International.

During the 10 minute fragrance/music treatment, participants were instructed to take off the cap of a sorbarod and sniff the fragrance as often as they wished. Initially, they were to think about the fragrance and the emotions that it brought and the scents that compose the fragrance. They were instructed to re-sniff the fragrance whenever they forgot or needed to refresh the fragrance image in their mind. While they were sniffing the fragrance, they were listening to the music using a personal CD player. Panelists were either sitting or lying down in a dimly lit room. During the bath exposure, panelists were provided with a fragranced bath product to use during the session. They were required to listen to a CD using a personal CD player during the bath. They also were required to dim the lights during their experience. The sensory procedure schedule was as follows: session 1 was 10 minutes of music and fragrance 30 minutes after waking; session 2 was 10 minutes of music and fragrance 4 hours after waking; session 3 was 10 minutes of music and fragrance 8 hours after waking; session 4 was 15 minutes of fragranced bath and music 12 hours after waking (or just prior to bedtime).

After one week and at the end of the two-week period, the participants were again evaluated for severity of acne. The results are shown in Tables 2 and 3.

TABLE 2
Reduction in Inflammatory Lesions

% Reduction In
Inflammatory Lesions

| Group Number | One Week | Two Weeks |
|---|---|---|
| 1 | 19 | 20 |
| 2 | 15 | 23 |
| 3 | 17 | 28 |

TABLE 3
Reduction in Total Lesions

% Reduction
In Total Lesions

| Group Number | One Week | Two Weeks |
|---|---|---|
| 1 | 12 | 15 |
| 2 | NS | 20 |
| 3 | NS | 12 |

NS = no significant change

The data demonstrates that the number of inflammatory lesions is reduced through the use of a skin cream with an anti-acne agent in it. Interestingly, it also appears that the addition of a relaxing fragrance, PD 1861 to the skin cream further reduced the number of lesions. The group that combined sensory experiences with the benzoyl peroxide skin cream demonstrated significant reduction in the number of inflammatory lesions after both the one and two week time points. Although, there appears to be less of a reduction in the total number of lesions after two weeks, it is important to note that inflammatory lesions are the most consumer noticeable symptom and product efficacy is often measured by the reduction in the number of inflammatory lesions.

It is interesting to note that there was a greater reduction of lesions on the second week of this study with the panelists who had some level of sensory treatment, either the use of a fragrance in the benzoyl peroxide skin cream, or the addition of relaxation elements to their regime. This is consistent with our hypothesis, as it would be expected that there would be lag time between the “relaxation” effects that would be present in the body, before they were manifested on the skin. The fact that there was a greater improvement for the second week in both of the cases where sensory was applied vs. the baseline skin cream supports this theory.

Participants were also asked to fill out an Emotional questionnaire before and after the study to determine if they felt the treatments made them feel better. The questions were as follows:
1. Overall, how would you describe your CURRENT PHYSICAL STATE?
   Grading was from 0 to 10, with 0 being “not at all relaxed” and 10 being “extremely relaxed”;
2. Overall, how would you describe your CURRENT ENERGY LEVEL?
   Grading was from 0 to 10, with 0 being “no energy” and 10 being “extremely energized”;
3. Overall, how would you describe your CURRENT EMOTIONAL STATE?
   Grading was from 0 to 10, with 0 being “not at all relaxed” and 10 being “extremely relaxed”;
4. Overall, how would you describe your CURRENT STRESS LEVEL?
   Grading was from 0 to 10, with 0 being “no stress at all” and 10 being “extremely stressed”.

The scores for each question before the study were compared to the scores for the same questions after the study. The data is reported in Table 4 as percent of score improvement after treatment based on the average score for the entire group of participants. The statistical significance for the data is in parenthesis.

TABLE 4
Emotional Questionnaire

| | Percent Of | | |
|---|---|---|---|
| | Score Improvement | | |
| Question | Group 1 | Group 2 | Group 3 |
| 1 | 8 (63) | 5 (72) | 37 (99) |
| 2 | 13 (83) | 5 (75) | 34 (96) |
| 3 | 9 (67) | 3 (63) | 43 (99) |
| 4 | −13 (95) | 3 (65) | 25 (97) |

Group 3 had a higher percent of participants feel better as a result of the treatment than any other group.

A second survey was performed before and after the study to establish if the participants felt the treatments were effective on their acne. The questionnaire that was used was the refined Skindex questionnaire that was published by Chren M, Lasek R, Flocke S Improved Discriminative and Evaluative Capability of a Refined Version of Skindex, a Quality-of-Life Instrument for Patients with Skin Diseases. Arch Dermatol. 1997; 133: 1433-1440 and is incorporated by reference. The statements were as follows:
1 My skin hurts;
2 My skin condition affects how well I sleep;
3 I worry that my skin condition may be serious;
4 My skin condition makes it hard to work or do hobbies;
5 My skin condition affects my social life;
6 My skin condition makes me feel depressed;
7 My skin condition burns or stings;
8 I tend to stay at home because of my skin condition;
9 I worry about getting scars from my skin condition;
10 My skin itches;
11 My skin condition affects how close I can be with those I love;
12 I am ashamed of my skin condition;
13 I worry that my skin condition may get worse;
14 I tend to do things by myself because of my skin condition;
15 I am angry about my skin condition;
16 Water bothers my skin condition (bathing, washing hands);
17 My skin condition makes showing affection difficult;
18 My skin is irritated;
19 My skin condition affects my interactions with others;
20 I am embarrassed by my skin condition;
21 My skin condition is a problem for the people I love;
22 I am frustrated by my skin condition;
23 My skin is sensitive;
24 My skin condition affects my desire to be with people;
25 I am humiliated by my skin condition;
26 My skin condition bleeds;
27 I am annoyed by my skin condition;
28 My skin condition interferes with my sex life;
29 My skin condition makes me tired.

The statements were scored as follows: 1=never; 2=rarely; 3=sometimes; 4=often; 5=all the time. The scores for each question before the study were compared to the scores for the same questions after the study. The data is reported in Table 5 as percent of score improvement after treatment based on the average score for the entire group of participants. The score changes are represented by those indicating changes in symptoms, function, emotions, and on an overall basis.

TABLE 5
Quality of Life Questionnaire

| | Percent Of Score Improvement | | | |
|---|---|---|---|---|
| | | Functional | | |
| | Symptoms | (questions 2, 4, | Emotional | |
| | (questions 1, 7, | 5, 8, 11, 14, 17, | (questions 3, 6, | |
| | 10, 16, 18, 23, | 19, 21, 24, 28, | 9, 12, 13, 15, 20, | |
| Group | 26,) | 29) | 22, 25, 27) | Overall |
| 1 | 2 | 8 | −3 | 4 |
| 2 | 1 | 2 | 8 | 5 |
| 3 | 4 | 8 | 12 | 9 |

The Group 3 participants had improved scores on the questionnaire than the other treatments. Positive effects from the combination of an anti-acne agent with a sensory regimen were seen symptomatically, functionally, emotionally, and on an overall basis.

### Example 3

### Other Treatment Regimes

Since there were clinical and perceived benefits of combining anti-acne skin creams with additional relaxation elements, additional studies were performed in an attempt to better delineate the relative contributions of both. In this separate study, Group 4 (13 panelists) applied benzoyl peroxide Skin Cream #3, which was identical to benzoyl peroxide skin cream #1, except the 6.67% of the 75% BPO was replaced with water. Group 5 (12 panelists), did not apply a benzoyl peroxide skin cream, but rather only participated in the identical Sensory regimen, as Group 3, which is listed below. The following protocol was used for Group #5: Group 5 smelled a fragrance PD1861 supplied by Quest International while sitting and listening to relaxing music from the music CD entitled “Relax with Ocean Relaxing Surf” by Eclipse Music Group for 10 minutes three times a day, and took a 35° C. 15 minute bubble bath at night containing fragrance PD1861 supplied by Quest International.

During the 10 minute fragrance/music treatment, participants were instructed to take off the cap of a sorbarod and sniff the fragrance as often as they wished. Initially, they were to think about the fragrance and the emotions that it brought and the scents that compose the fragrance. They were instructed to re-sniff the fragrance whenever they forgot or needed to refresh the fragrance image in their mind. While they were sniffing the fragrance, they were listening to the music using a personal CD player. Panelists were either sitting or lying down in a dimly lit room. During the bath exposure, panelists were provided with a fragranced bath product to use during the session. They were required to listen to a CD using a personal CD player during the bath. They also were required to dim the lights during their experience. The sensory procedure schedule was as follows: session 1 was 10 minutes of music and fragrance 30 minutes after waking; session 2 was 10 minutes of music and fragrance 4 hours after waking; session 3 was 10 minutes of music and fragrance 8 hours after waking; session 4 was 15 minutes of fragranced bath and music 12 hours after waking (or prior to bedtime).

After one week and at the end of the two-week period, the participants were again evaluated for severity of acne using the modified Cook Global Acne Grading Scale described above. The results are shown in Tables 6 and 7.

TABLE 6
Reduction in Inflammatory Lesions

% Reduction In
Inflammatory Lesions

| Group Number | One Week | Two Weeks |
|---|---|---|
| 4 | 36 | 50 |
| 5 | 23 | 28 |

TABLE 7
Reduction in Total Lesions

% Reduction
In Total Lesions

| Group Number | One Week | Two Weeks |
|---|---|---|
| 4 | 29 | 33 |
| 5 | 14 | 7 |

The placebo Group 4 clearly improved significantly in terms of reduction of inflammatory lesions. This is thought to be at least partially due to improved hygiene during the study, the consumer expectation of the “treatment”, and potentially to the benzoyl peroxide Skin Cream #3 itself. This is somewhat shown by the fact that the use of the relaxation elements by themselves show dramatic reductions in the number of inflammatory lesions. It is important to note that the study in Example 3 was performed at a different time of the year than Examples 1 and 2, so the results of Example 3 should not be compared to the results of Examples 1 and 2. As with the data relating to inflammatory lesions, the data for reduction in total number of lesions also showed that both treatments were effective, with the benzoyl peroxide skin cream #3, being more effective.

Groups 4 and 5 also answered the Emotional and Skindex questionnaires described above about the ability of the treatments to make them feel better. Their results are listed in Tables 8 and 9 below.

TABLE 8
Emotional Questionnaire

| | Percent Of | |
|---|---|---|
| | Score | |
| | Improvement | |
| Question | Group 4 | Group 5 |
| 1 | 13 (97) | −8 (58) |
| 2 | −1 (66) | 3 (88) |
| 3 | 8 (70) | 7 (84) |
| 4 | 15 (87) | 11 (90) |

TABLE 9
Quality of Life Questionnaire

Percent Of
Score Improvement

| Group | Symptoms | Functional | Emotional | Overall |
|---|---|---|---|---|
| 4 | 1 | 4 | 10 | 6 |
| 5 | 0 | 10 | 6 | 7 |

These tables show that the relaxation treatment by itself showed broad emotional and well-being benefits to the panelists in this study. This is consistent with the clinical improvement that was seen in the panelist's acne condition. Improvements were also observed for Group 4, however, they did not seem to be as high as commensurate with the clinical improvement observed. Clearly, the optimal treatment for this condition combines both clinical and psychological benefits.

Since one of the factors that lead to acne is an increased amount of sebum, sebum production was measured for Groups 4 and 5 as well. It has been postulated that an increase in sebum could result after an increase in stress activity through an adrenocorticotrophic hormone pathway. An increase in sebum production could be related to an increase in the visual appearance of shine. The results are listed in Table 10.

TABLE 10
Effect of Treatments on Sebum reduction

| Study Cell | Week 1 Reduction | Week 2 Reduction |
|---|---|---|
| Group 4 | 26% | −2% |
| Group 5 | 16% | 8% |

Once again, the benefits for sebum reduction are observed in the cell with the relaxation elements in them. To further confirm this hypothesis, measures of stress markers were taken to determine changes in stress levels throughout the study.

Some participants were also asked to collect approximately 1 ml of saliva by drooling or spitting into independent vials at set points throughout each day of the study for the purpose of measuring cortisol concentrations. Area under the curve measurements were taken as described in co-pending patent application “Methods For Measuring Stress In Mammals”60/256,812 filed Dec. 20th 2000 The area under the curve prior to the study was compared to the area under the curve after treatment. The results are shown in Table 11 as percent decrease in area under the curve (reduction in baseline cortisol levels).

TABLE 11

Percent Reduction In Cortisol
Area Under The Curve

| Treatment | One Week | Two Weeks |
|---|---|---|
| Group 4 | 19 | 5 |
| Group 5 | 38 | 23 |

Group 5 had a decreased baseline cortisol level within one week, which lasted through the second week. This result correlated with the consumers' self-assessment, as well as the clinical data that was obtained. Separately, Group 4 (which had no inherent relaxation treatments beside the knowledge that the panelists were on a “treatment” study) showed a decrease in cortisol levels after one week. While a decrease was still seen after two weeks, the effect was not as sustained as was for Group 5 (having participated in the sensory regimen).

While we have shown that the reduction of cortisol has led to improved skin condition in the case of acne, this should also be effective for the treatment of other stress-related skin diseases such as: atopic dermatitis, sebhorreic dermatitis, psoriasis, itch, wound healing, fine lines and wrinkles, pigmentation, etc.

1-53. (canceled)

54. A method for improving the efficacy of an anti-acne composition said method comprising the step of administering a sensory regimen in an amount effective to downregulate the activity of the HPA axis of said mammal in combination with the administration of said anti-acne composition comprising an anti-acne agent; wherein said sensory regimen is selected from the group consisting of auditory stimuli, visual stimuli, tactile stimuli, gustatory stimuli and olfactory stimuli, and combinations thereof and is effective to downregulate the activity of the HPA axis and wherein said activity of the HPA axis is downregulated by at least one of the following methods: (1) reducing the amount of total daily adrenocortical hormone; (2) reducing adrenocortical hormone at any time point in the period from about 4 to about 8 hours following morning waking; (3) reducing the total daily adrenocortical hormone minus the integrative measure of morning peak adrenocortical hormone.

55. A method according to claim 54, wherein the anti-acne composition is administered orally or topically.

56. A method according to claim 54, wherein the anti-acne composition is administered topically and the anti-acne agent is selected from salicylic acid, sulfur, lactic acid, glycolic acid, pyruvic acid, urea, resorcinol, N-acetylcysteine, retinoic acid, benzoyl peroxide, octopirox, triclosan, azelaic acid, phenoxyethanol, phenoxypropanol, flavinoids, derivatives thereof, and mixtures thereof.

57. A method according to claim 56, wherein the anti-acne agent is selected from benzoyl peroxide, salicylic acid, and mixtures thereof.

58. A method according to claim 55, wherein the anti-acne agent is a pharmaceutical compound selected from isotretinoin and tretinoin, adapalene, tazarotene, azelaic acid, minocycline, doxycycline, erythromycin and clindamycin and mixtures thereof.

59. A method according to claim 55, wherein the anti-acne composition is administered orally and is isotretinoin.

60. A method according to claim 55, wherein the anti-acne composition further comprises an antibacterial agent.

61. A method according to claim 54, wherein the sensory regimen comprises olfactory stimuli provided by an effective amount of a relaxing fragrance present in the anti-acne composition.

62. A method according to claim 61, wherein the sensory regimen further includes visual stimuli comprising soft lighting.

63. A method according to claim 62, wherein the sensory regimen further includes auditory stimuli comprising music.

64. A method according to claim 54, wherein the sensory regimen is administered daily for at least one week.

65. A method according to claim 64, wherein said sensory regimen comprises olfactory stimuli comprising soaking in a bath fragranced with a relaxing fragrance in combination with auditory stimuli comprising listening to relaxing music.

66. A method according to claim 64, wherein the sensory regimen further includes visual stimuli comprising soft lighting.

67. A method of improving the quality of life of an individual by use of the method of claim 54.

68. An improved method for reducing the number and severity of acne lesions comprising the administration of an anti-acne composition, said improvement comprising the administration of a sensory regimen in an amount effective to downregulate the activity of the HPA axis of said mammal in combination with said administration of said anti-acne composition; wherein said sensory regimen is selected from the group consisting of auditory stimuli, visual stimuli, tactile stimuli, gustatory stimuli and olfactory stimuli, and combinations thereof and is effective to downregulate the activity of the HPA axis and wherein said activity of the HPA axis is downregulated by at least one of the following methods: (1) reducing the amount of total daily adrenocortical hormone; (2) reducing adrenocortical hormone at any time point in the period from about 4 to about 8 hours following morning waking; (3) reducing the total daily adrenocortical hormone minus the integrative measure of morning peak adrenocortical hormone.

69. A method of improving the quality of life of an individual by use of the method of claim 68.

70. A method according to claim 68, wherein the anti-acne composition is administered orally or topically.

71. A method according to claim 68, wherein the anti-acne composition is administered topically and the anti-acne agent is selected from salicylic acid, sulfur, lactic acid, glycolic acid, pyruvic acid, urea, resorcinol, N-acetylcysteine, retinoic acid, benzoyl peroxide, octopirox, triclosan, azelaic acid, phenoxyethanol, phenoxypropanol, flavinoids, derivatives thereof, and mixtures thereof.

72. A method according to claim 71, wherein the anti-acne agent is selected from benzoyl peroxide, salicylic acid, and mixtures thereof.

73. A method according to claim 70, wherein the anti-acne agent is a pharmaceutical compound selected from isotretinoin and tretinoin, adapalene, tazarotene, azelaic acid, minocycline, doxycycline, erythromycin and clindamycin and mixtures thereof.

74. A method according to claim 70, wherein the anti-acne composition is administered orally and is isotretinoin.

75. A method according to claim 70, wherein the anti-acne composition further comprises an antibacterial agent.

76. A method according to claim 68, wherein the sensory regimen comprises olfactory stimuli provided by an effective amount of a relaxing fragrance present in the anti-acne composition.

77. A method according to claim 76, wherein the sensory regimen further includes visual stimuli comprising soft lighting.

78. A method according to claim 76, wherein the sensory regimen further includes auditory stimuli comprising music.

79. A method according to claim 68, wherein the sensory regimen is administered daily for at least one week.

80. A method according to claim 79, wherein said sensory regimen comprises olfactory stimuli comprising soaking in a bath fragranced with a relaxing fragrance in combination with auditory stimuli comprising listening to relaxing music.

81. A method according to claim 80, wherein the sensory regimen further includes visual stimuli comprising soft lighting.

82. A method according to claim 68, wherein the reduced activity of the HPA axis results in a reduction in adrenocortical hormone levels of said mammal.
